(19) United States
(12) Patent Application Publication
(10) Pub. No.: US 2017/0172450 A1
(43) Pub. Date: Jun. 22, 2017

(54) SYSTEM AND METHODS FOR QUANTITATIVELY DESCRIBING BIOPHYSICAL MARKERS

(71) Applicants: STC.UNM, Albuquerque, NM (US); Board of Regents, The University of Texas System, Austin, TX (US); The Methodist Hospital, Houston, TX (US)

(72) Inventors: Eugene KOAY, Houston, TX (US); Vittorio CRISTINI, Albuquerque, NM (US); Jason FLEMING, Houston, TX (US); Priya BHOSALE, Bellaire, TX (US); Christopher CRANE, Houston, TX (US); Haifa SHEN, Bellaire, TX (US); Mauro FERRARI, Houston, TX (US)

(21) Appl. No.: 15/123,867

(22) PCT Filed: Mar. 6, 2015
(86) PCT No.: PCT/US15/19127

§ 371 Date: Sep. 6, 2016

Related U.S. Application Data
(60) Provisional application No. 61/949,327, filed on Mar. 7, 2014.

(51) Int. Cl. A61B 5/055; A61B 6/03; A61B 6/00; A61B 5/00

(52) U.S. Cl. CPC A61B 5/055; A61B 5/425; A61B 5/4842; A61B 5/4848; A61B 6/032; A61B 6/481; A61B 6/5217; A61B 6/12

(57) ABSTRACT

Disclosed are method of detecting or characterizing a tumor by quantitatively measuring and comparing changes in mass transfer into the tumor and normal tissue over time using computed tomography or magnetic resonance imaging. The methods can be used to predict therapeutic response to treatment and to develop treatment plans.

### CROSS REFERENCE TO RELATED APPLICATIONS

This application claims the benefit of U.S. Provisional Application No. 61/949,327, filed Mar. 7, 2014, which is incorporated by reference in its entirety.

### STATEMENT REGARDING FEDERALLY SPONSORED RESEARCH

This invention was made with government supported under grant NCT01276613 awarded by the Lustgarten Foundation, and grant U54CA143837, awarded by the National Cancer Institute (NCI). The United States Government has certain rights in the invention.

### FIELD OF THE INVENTION

The invention is directed to a system and methods for quantitatively describing biological markers. More specifically, the invention uses diagnostic radiology scans, such as computed tomography (CT) or magnetic resonance imaging (MRI), to quantitatively describe physical properties of tissues.

### BACKGROUND OF THE INVENTION

Pancreatic ductal adenocarcinoma (PDAC) is one of the most lethal malignancies, and decades of research have yielded minimal improvement in the median overall survival of patients. Although several biological explanations have been proposed for the poor therapeutic outcomes of patients, physical phenomena may also contribute to the significant difficulty in treating this disease. Pre-clinical and clinical investigations have suggested that the therapeutic resistance associated with PDAC may be partly attributed to ineffective chemotherapy delivery to cancer cells.

Solid tumors, such as PDAC, often have mass transport properties that differ from those of normal tissues. PDAC exhibits several pathological features that can be considered physical barriers to effective drug delivery, including disorganized, leaky, and non-functional vasculature, characteristically dense stroma, and deregulated cellular transport proteins. Leaky vasculature of solid tumors can create high interstitial fluid pressure, preventing movement of chemotherapeutic agents from the vasculature to the extracellular compartment. The extracellular compartment of PDAC is usually a dense stromal (desmoplastic) reaction that has been shown to influence the delivery of and response to gemcitabine in pre-clinical models. Indeed, differential mass transport has allowed qualitative differentiation between normal and pathological pancreatic tissues. The pancreatic protocol computed tomography (CT) scan is a diagnostic test that exemplifies this differential mass transport principle. PDACs are typically hypodense compared with the normal pancreas during the timed phases of the diagnostic test.

Altered mass transport is also affected by cellular and molecular properties. Human equilibrative nucleoside transporter (hENT1) is the primary transport protein by which gemcitabine and other nucleoside analogues enter the cellular compartment and ultimately inhibit DNA replication. Expression of hENT1 human PDAC varies among individual tumors, and the expression level of hENT1 has been shown to correlate with outcome after adjuvant gemcitabine therapy, presumably due to differential cellular uptake of gemcitabine due differential expression of functional hENT1.

Frequently, response to chemotherapeutic agents is evaluated empirically. Administration of a chemotherapeutic agent that ultimately proves to be ineffective in treating the cancer is undesirable because such treatments are expensive and associated with significant unpleasant side effects that can compromise a patient's quality of life. Further, in the case of a cancer for which alternative treatments are available, effective treatment is delayed.

There exists a need for improved methods of characterizing cancer to assist in the development of therapeutic strategies. The present invention addresses that need.

### SUMMARY OF THE INVENTION

In certain embodiments, the invention is a system and methods that quantitatively describe a physical property of a tissue in a subject. The method includes administering a contrast material to the subject. Radiological scans of the tissue are obtained at various time points, including before and after administration of the contrast material. The scans are then used to obtain a plurality of density measurements, and the density measurements are evaluated to detect changes in density over time.

The invention can be used to identify and amplify differences between normal and abnormal tissues on routine diagnostic scans, for example computed tomography (CT) scans or magnetic resonance imaging (MRI) scans. In certain embodiments, model parameters differentiate normal tissue from tumor tissue by a several-fold difference between normal and malignant tissues.

In certain embodiments, the methods of the invention can use the model parameters to predict chemotherapy delivery to cancer cells.

In certain and embodiments, the methods of the invention can use the model parameters to predict tumor response to chemotherapy and radiation.

In certain embodiments, the invention can be used to measure changes in mass transport properties after therapies, which may relate to failure patterns, i.e., local versus distant failure patterns such as after chemoradiation. These parameters represent physical biomarkers that may have prognostic or predictive value for clinicians and patients.

Specifically, an important advantage of the invention over routine diagnostic scans currently in use is that it provides quantitative and clinically meaningful diagnostic and prognostic information to clinicians and patients that routine diagnostic scans do not provide. Other diagnostic imaging modalities that quantify mass transport would require another test beyond the routine diagnostic scan with added risks to the patient and additional cost. The quantitative data that the invention provides is patient-specific, allowing for a personalized approach to medical therapy. This type of physical biomarker previously did not exist to aid clinicians in the manners described.

For purposes of this application, the present invention is discussed in reference to pancreatic cancer and esophageal cancer. However, the present invention is applicable to any solid tumor as well as other human conditions in which mass transport may be altered.

In certain embodiments, the system and methods obtain information about a physical biomarker derived from patient diagnostic radiology scans. The standard CT for pancreatic cancer workup and diagnosis is a “pancreatic protocol”. This protocol usually entails four phases: precontrast, arterial, portal-venous, and delay. The four phases are timed in relation to the injection of intravenous contrast. As the contrast enters the tissue, density changes are detected by a CT scanner. These are quantified by the Hounsfield Unit (HU).

Measuring the HU of the normal pancreas and pancreatic tumor at each phase of the pancreatic protocol provides a measurement of the difference between normal and cancerous pancreatic tissue. Indeed, this was the objective when the pancreatic protocol was designed: to differentiate pathology from normal tissue based on differential uptake of contrast so that the human eye could detect the differences.

The initial time point is the precontrast phase. The next time point (t=35-40 seconds (s) after contrast injection) is the arterial phase, the timing of which can vary from one institution to the next. The subsequent time point is the portal-venous phase (t=65-70 s after contrast injection), which can also vary from one institution to the next. Finally, the delay phase occurs at 3 to 5 minutes after the injection of contrast (this timing can also vary across institutions).

The invention is a system and methods that takes the data obtained from the above pancreatic protocol and uses a mathematical model to quantify mass transport properties of the tissue in question, based on the HU measurements from each phase of the protocol.

The transport model consists of one ordinary differential equation describing the variable density y(t) (HU) in the tissue as a function of time t resulting from transfer of contrast agent molecules through the vessel walls at a rate r (s⁻¹):

$$\frac{dy}{dt} = r \cdot {( {Y}_{max} \cdot {e}^{- {r}_{c} t} - y )}_{i} \qquad Eq . ( 1 )$$

where the first term within parentheses represents the (imposed) level of density within the micro-vasculature. Equation (1) is solved for intimal condition y(0)=0 to lead the solution for the variable density:

$$y ( t ) = {Y}_{max} \cdot r \cdot \frac{{e}^{- {r}_{c} t} - {e}^{- rt}}{r - {r}_{c}} \qquad Eq . ( 2 )$$

The three phenomenological model parameters Yₘₐₓ (HU), r and r_{c} (s⁻¹) describe: the maximum value of density (i.e., at t=0) measurable in the micro-vasculature of the tissue region of interest, the rate of transfer of contrast agent from the micro-vasculature to the tissue, and the rate of clearance of the contrast agent from the micro-vasculature. Thus Yₘₐₓ and r, represent the qualities of transport within the microvessels, while r_{c} characterizes extravasation and transport into (and away from) the tissue.

The model parameters were estimated for each patient by performing least square fits of the solution of the model for the variable density Eq (2) to the four time-point CT measurements at t=0 (precontrast), t=40 s (arterial), t=70 s (venous), and t=300 s (delay), all in HU. Characteristic average values thus calculated across the patients' ensemble were: Yₘₐₓ≈100 HU, r≈0.1 s, and r_{c}≈0.001 s. Two important additional variables can be derived from the mathematical model. It should be noted that neither of these can actually be measured. The first one is the initial “slope” of the predicted tissue density profile, i.e.:

at t=0, dy/dt=s₀=Yₘₐₓr, Eq. (3a)

i.e., the initial time derivative. The second one is the predicted “maximum value” of density attained within tissue (which may happen for t>300 s, and thus outside of the time-window of the CT measurements):

$$at t = log ( r \text{/} {r}_{c} ) \text{/} ( r - {r}_{c} ) , dy \text{/} dt = 0 , and \\ y = {y}_{max} = {Y}_{max} \cdot {( r / {r}_{c} )}^{- \frac{{r}_{c}}{r - {r}_{c}}} \qquad Eq . ( 3 b )$$

It should be noted that, as expected, for all t, y(t)≦yₘₐₓ<Yₘₐₓ, i.e., the value of density in the tissue cannot exceed the initial amount delivered to the micro-vasculature from the aorta.

The present invention and its attributes and advantages will be further understood and appreciated with reference to the detailed description below of presently contemplated embodiments.

### BRIEF DESCRIPTION OF THE DRAWINGS

FIG. 1 is a diagram showing the steps of a method for measuring mass transport in PDAC.

FIG. 2 illustrates the application of a method to measure mass transport properties from the CT scans of two patients with different enhancement profiles.

FIG. 3 shows a simple linear piece-wise function can be used to estimate the AUC from the CT scans.

FIG. 4 is a plot showing the correlation between normalized AUC and the amount of stroma measured on the pathology specimens of patients with PDAC.

FIG. 5 is a plot showing the correlation between normalized AUC and the amount of gemcitabine incorporated into the DNA of cells within PDAC tumors.

FIG. 6 is a plot showing the correlation between normalized AUC and the pathological response to cytotoxic therapy.

FIG. 7 is a plot of overall survival of patients with resectable PDAC with high or low AUC.

FIG. 8 illustrates relationship of normalized AUC ratios and treatment outcomes in patients with locally advanced PDAC.

FIG. 9 shows the association between normalized AUC ratios and local progression free survival in patients with locally advanced PDAC.

FIG. 10 illustrates measuring normalized AUC for esophageal cancers and other instances when only a single time point for measuring the HU in the tissue is available.

### DETAILED DESCRIPTION OF EMBODIMENTS OF THE INVENTION

Although other cancers have seen significant improvements in overall survival over the past several decades, PDAC has continued to have a poor prognosis. Advances in the understanding of the genetics and biology of PDAC have led to novel therapeutic strategies, but applications of these strategies in PDAC have not resulted in improved clinical outcomes for patients. One of the challenges that may influence outcome is delivery of the drug to its molecular target, but the concept of mass transport characterization in human cancer has not been integrated into clinical practice.

Described is the development, validation, and application of methods to quantify mass transport for individual human PDAC tumors. The data support the concept that mass transport phenomena influence the delivery of and response to gemcitabine-based therapies. These CT analysis methods have wide clinical applications for diagnostic and therapeutic planning, as the principles of mass transport can be applied to any human pathological process as well as a variety of therapeutic agents. The clinical trial design of intraoperative drug infusion is an important component in the study of mass transport in PDAC, represents a novel platform to study mechanisms of targeted drug delivery, and complements the CT analysis. Combined, the methodologies described and the results obtained may lead to rational interventions for pancreatic cancer and other solid tumors that improve drug delivery and thereby extend survival for patients.

The CT analysis can be integrated into existing standard-of-care diagnostic tests, such as the pancreatic protocol CT that is ubiquitously used clinically. Others have used dynamic contrast enhanced magnetic resonance imaging (DCE MRI) and perfusion CT to derive mass transport properties from imaging, correlating these properties with response to cancer therapy. In contrast to mass transport analysis of the pancreatic protocol CT, DCE MRI and perfusion CT require an additional imaging test after the routine scan is performed, resulting in increased contrast exposure to the patient and additional expense. The CT transport analysis developed can be integrated into the existing pancreatic protocol CT, enhancing it for applications beyond its current capabilities.

For instance, the parameters that are derived from the mass transport model describe qualities of the tissue of interest and its surrounding vasculature, and these parameters quantitatively differentiate malignant from normal tissue processes by at least 2 fold. This provides a stark contrast between these tissues and offers evidence that mass transport is diminished or limited in pancreatic tumors compared to normal pancreas tissue. With further development, these quantitative differences in mass transport may help radiologists more clearly distinguish between normal and pathological processes. The methods provide valuable information for use in therapeutic-planning, in which pre-treatment biophysical characterization of a patient's tumor is used to inform management decisions.

As described in the Examples, the CT-derived parameter normalized AUC correlated with gemcitabine incorporation, pathological response, and survival after gemcitabine-based therapies. It is notable that other model parameters also showed correlations, but AUC was found to be the most robust parameter identified to date. Specifically, AUC, by its definition as an integral over time, is less sensitive to minor variations in the CT acquisition and patient physiology compared to other model parameters. The finding that the model parameter AUC can be approximated in a simple and accurate manner means that the technique can be performed at any institution without any complex algorithms or software as long as the HU of the pancreatic tissues can be measured and the timing is known for each sequence of the pancreatic protocol CT scan, which is currently readily available.

Further, the AUC may be used as a quantitative surrogate for stromal score, which suggests that the CT parameters have a histopathological basis. Both the CT parameters and the stromal score (after accounting for hENT1) were inversely correlated with gemcitabine DNA incorporation into the tumor. On the other hand, in the context of neoadjuvant chemoradiation for potentially resectable PDAC, patients with tumors with higher normalized AUC were found to have better responses and outcomes. One possible explanation is that tumors with higher stromal scores may have more abnormal vasculature; hence these tumors with more stromal reaction may exhibit arterial-venous shunting. This could manifest as higher values of transport parameters such as AUC but lower levels of chemotherapy delivery because of mismatch between convective and diffusion-based transport of gemcitabine, preventing the cells from exposure to drug. Despite wide variability, it is notable that there was measureable drug delivery in all patients in the clinical trial of intraoperative gemcitabine infusion. It is possible that any drug delivery may be enough to sensitize cells to radiation, and radiation is likely the main determinant of response in the context of neoadjuvant chemoradiation. Moreover, it is possible that greater enhancement may correlate with better oxygenation, an important factor in radiation sensitivity.

Data from the clinical trial of intraoperative drug infusion in 12 patients support the multi-scale transport hypothesis. First, a correlation between CT-derived vascular properties and gemcitabine incorporation was observed. Second, the data demonstrate evidence that the stroma impairs drug delivery by acting as a physical barrier to chemotherapy delivery or by disrupting vascular function, as tumors with higher stromal scores had lower gemcitabine DNA incorporation, when hENT1 expression levels were taken into account

There was overlap in the tumor gemcitabine incorporation between patients with low and high hENT1 scores. Of note, one of the seminal studies of hENT1 in PDAC demonstrated an association between hENT1 expression and outcome, but not delivery. That study was in the context of adjuvant gemcitabine, i.e., involved cases in which the primary tumor and its stroma had been removed, indicating that chemotherapy effected microscopic cancer cells. The sequential influence of the primary tumor's multi-scale transport processes on the delivery of chemotherapy to cells would not apply to the adjuvant setting. Interestingly, hENT1 level alone does not correlate with prognosis in the neoadjuvant setting, also supporting the multi-scale transport hypothesis. The data are consistent with previous pre-clinical work and clinical observations, but are unique in that they are the first demonstration that drug delivery in PDAC may depend on multi-scale transport phenomena. Considering the context of previous clinical studies of hENT1, the results show that a single molecular biomarker (i.e., low or high hENT1) is not sufficient to select and enrich for those patients who would benefit from novel gemcitabine formulations in the primary disease setting. Furthermore, these data strongly support the Transport Oncophysics concept of multi-scale mass transport deregulation (e.g., vascular, extracellular, and cellular) as a hallmark of cancer, and illustrate how these transport phenomena may be used to individualize clinical cancer management. It is envisioned that pre-therapy CT imaging analysis can predict gemcitabine incorporation.

Toward the goal of individualization, the unique clinical trial platform described can be used to study biological, pathological, and physical correlates of drug delivery in humans. Others have evaluated drug delivery in humans during therapy or measured transport-related changes after chemotherapy. In contrast, the invention describes mass transport properties of a tumor and how those properties influence drug delivery. In the development of this clinical trial, extensive calibration, validation, and correlative studies were performed. An understanding of factors influencing drug delivery in humans with tumors, such as PDAC, promotes developments of rational interventions with improved therapeutic outcomes. In certain embodiments, the intraoperative drug infusion clinical trial platform can be used to test methods to alter the physical environment of the tumor, thereby increasing drug delivery and providing rationale for future clinical trials that aim to improve outcomes with these strategies.

Along these lines, the data indicate that some tumors may have physical properties that respond well to chemoradiation. For example, it was discovered that normalized AUC correlates with overall survival. It is conceivable that these physical properties are related to underlying biological processes. By investigating how the underlying molecular biology of the tumor and host may affect these biophysical signatures, targeted therapies, including emerging companion diagnostics, that modify the biophysical environment (stroma) of the tumor could be used to alter and track the properties of tumors for improved delivery and efficacy of systemic agents, shifting the biophysical profile along the observed response relationship. This targeting of “physical resistance” could complement targeting of “biological resistance,” and, in some instances, may be one and the same.

Considering their robust correlations with gemcitabine incorporation, pathological response, and oncologic outcome, CT-derived mass transport parameters represent biophysical markers that have significant implications for cancer medicine. Further development of diagnostic tests that simultaneously allow radiologic cancer staging as well as biophysical tumor profiling is warranted. The concept of individually tailored cancer therapy based on biophysical characterization is also supported by this work, as patients with good response to therapy appear to have different physical properties compared to those with poorer responses. The clinical trial platform of intraoperative drug infusion during resection suggests that the sequential contributions of vascular, extracellular, and cellular transport influence gemcitabine incorporation. The system and methods of the invention may be used to further study these transport mechanisms and to develop rational therapeutic interventions for patients. In summary, this invention allows quantitation of biophysical markers that help direct cancer treatment and improve the survival of patients with PDAC and other solid tumors.

The examples below reveal considerable inter-patient and intra-tumoral heterogeneity in the delivery of gemcitabine and in the mass transport properties of pancreatic cancer. This heterogeneity can be reproducibly described with the volumetric mass transport methodology described herein to analyze the CT scans. This volumetric measurement of mass transport uses multiple regions of interest. The invention uses mass transport properties in patient stratification and therapeutic guidance in treating malignancies such as PDAC.

Biophysical phenomena interact with biological properties of pancreatic cancer to influence heterogeneous drug delivery, as both the CT-derived properties and hENT1 score influenced correlations and their degree of accuracy. The Transport Oncophysics view of multi-scale transport dysregulation is a key feature of cancer, i.e., that the heterogeneity in drug delivery may be due to local differences in the physical microenvironment (e.g., microvascular density, collagen and stromal content) and expression of cellular transporters of gemcitabine (e.g., hENT1). The proposed mechanism of heterogeneous drug delivery in human pancreatic cancer is also supported by the clinical observation of heterogeneity in the histology of the specimens. For example, the stromal score varied from 10 to 60% in the 12 patients.

It is likely that the variable drug delivery observed in pancreatic cancer impacts therapeutic outcome. As has been shown through a mechanistic mass transport model of cell kill by chemotherapy, local physical properties of the tumor can describe pathological response in the immediate vicinity. Furthermore, this mechanistic model supports the idea that response is closely tied to drug delivery: more predicted drug delivery translated to more tumor cell kill. Combined, these results indicate that heterogeneity in the pathological response of tumors is due to variations in the biophysical properties that influence drug delivery across the tumor.

Few studies have assessed drug delivery in human cancers, but the observations from these studies appear to consistently demonstrate substantial heterogeneity. For example, for patients with squamous cell carcinomas of the head and neck, gemcitabine incorporation into the primary tumors demonstrated up to 5 fold differences at a dose of 300 mg/m². However, the mechanisms of heterogeneity in the drug delivery were not explored in this Phase I trial. Regional variations in doxorubicin delivery were also shown in breast cancer. There was significant intra-tumoral heterogeneity in delivery within the breast tumors of the patients, with some parts of the tumor not receiving any drug.

In the current study, heterogeneity in delivery between patients and within pancreatic tumors was observed. Notably, the Advion measurement of gemcitabine DNA incorporation has high inter-assay precision and accuracy, making it unlikely that the variability in gemcitabine DNA incorporation was due to the measurement technique. The true extent of heterogeneity may not be known, as the designations of outer and inner tumor were defined only by visual inspection and palpation. These data are informative in guiding future efforts to understand drug delivery in cancers, such as pancreatic cancer. The methods of the invention may derive transport properties from CT scans for use in predicting drug delivery to tumors. It is conceivable that the variability in drug delivery is directly related to the clinical observation of variable response to treatment in pancreatic cancer. By combining the principles of mass transport with characterization of the molecular drivers of pancreatic cancer, individualized approaches to patients may be achieved to improve outcomes.

Applying the model according to the invention to patient CT scans, a stark difference can be seen between normal and cancerous tissues in terms of the magnitude of the derived parameters. There is sharp separation in the distribution of values of the mathematical model parameters for pancreatic lesions and those for normal pancreatic tissue, where the parameter values are lower by at least two-fold in the tumors, enhances the ability to differentiate normal tissue from malignant tissue. Thus, in certain embodiments, the system and methods provide allow detection and diagnosis of malignancies having altered mass transport.

The invention also predicts drug delivery, response and outcome. With application of the model in drug delivery, the contrast can be thought of as a surrogate for the drug. In this way, a clinician could predict how much drug would be delivered to the target tissue prior to prescribing the drug. Even though the drug and contrast molecules may differ in circulating times, drug half-lives, cellular uptake, metabolism, and other factors, the mathematical model parameters correlate with the gemcitabine incorporation into the tumor. Further mathematical model refinements to account for differences between the drug of interest and the contrast material can be developed. As a demonstration of the drug application for the invention, in this “Phase 0” experiment gemcitabine was intravenously infused into patients during curative resection of localized pancreatic cancer. The gemcitabine incorporation into the DNA of the tumor and normal pancreas cells was measured using a commercial assay. The significant correlation between the model parameters and gemcitabine incorporation indicates that the model parameters can be used to predict how much drug can be delivered to tissues. With this knowledge, one could possibly predict who would most benefit from chemotherapy and who might not benefit. Additionally, one may also predict the magnitude of the benefit based on the delivery amount given by the model and perhaps other molecular biomarkers.

The invention also can be used to quantify mass transport properties prior to preoperative chemotherapy and chemoradiation for localized cancer such as pancreatic cancer and esophageal cancer. These mass transport properties correlate with the pathological response to therapy and oncologic outcome. The patients with a near-complete response to preoperative therapy have unique mass transport properties compared to those who have less than a near-complete response. Furthermore, those with a near-complete response have longer cancer-specific survival than those without a near-complete response. Thus, the system and methods can be used to identify those who may or may not benefit from the toxicities of preoperative chemotherapy and chemoradiation for localized pancreatic cancer and esophageal cancer.

The invention also detects changes in physical signatures in response to therapy and predicting outcome. The changes in the model parameters after chemotherapy and chemoradiation for locally advanced pancreatic cancer indicate that the tissues have changed their mass transport properties. This change in mass transport represents a physical biomarker that can be used to predict the outcomes of the patient. Specifically, there is quantitative separation in the physical biomarker for patients who had progression in the pancreas after therapy (termed local failure) and patients who had progression outside the pancreas after therapy (termed distant failure). This is clearly detectable from the several-fold separation in the peaks of the two corresponding distributions. The correlation of this physical biomarker with patient outcome can provide valuable prognostic information for clinicians and patients.

In certain embodiments, the invention involves using a calculation that approximates parameters derived from the mathematical model to describe the time-dependent changes in density in pancreatic tissues during contrast enhanced CT scans. The pancreatic protocol involves well-timed scans in relation to contrast injection: pre-contrast, arterial phase, and portal-venous phase. Systematic measurements of the pancreatic tumor and normal pancreas were recorded, and a model was developed to derive transport properties from these measurements. The model function can be integrated with time to derive an area under the curve (AUC), and a simple piece-wise linear function can be used to estimate AUC.

In certain embodiments, the simple piece-wise function is calculated as follows:

1. Calculate the AUC of the Pancreas

AUC_{pancreas}=tₐᵣₜₑᵣᵢₐₗ*HU_{pancreas@arterial}+tₚₒᵣₜₐₗ₋ᵥₑₙₒᵤₛ*(HU_{pancreas@arterial}+HU_{pancreas@portal-venous})

2. Calculate the AUC of the Pancreatic Tumor

AUCₜᵤₘₒᵣ=tₐᵣₜₑᵣᵢₐₗ*HU_{tumor@arterial}+tₚₒᵣₜₐₗ₋ᵥₑₙₒᵤₛ*(HU_{tumor@arterial}+HU_{tumor@portal-venous})

3. Calculate the Estimated Normalized AUC

Estimated normalized AUC=AUCₜᵤₘₒᵣ/AUCₚₐₙₒᵣₑₐₛ
where HU is the density of the tissue at a specific time point of the CT acquisition (arterial or portal-venous), tₐᵣₜₑᵣᵢₐₗ is the time of the arterial phase acquisition defined by the model (˜5 s at MD Anderson) and tₚₒᵣₜₐₗ₋ᵥₑₙₒᵤₛ is the time of the portal-venous phase acquisition defined by the model (˜35 s at MD Anderson).

The estimated normalized AUC was calculated using the simple piece-wise linear equation was compared with the model-derived normalized AUC. There is a 1:1 correlation between the estimated and model-derived values, indicating that one of the model parameters can be accurately assessed without need for the original mathematical model. This makes the technology easily exportable and simple to use at any institution.

In certain embodiments, changes in mass transport are used to assess response of pancreatic cancer to cytotoxic therapies. A decrease in tumor enhancement on CT scans after cytotoxic therapy is generally regarded as a good response. In certain embodiments, measuring this phenomenon can be used to assess local control of human pancreatic adenocarcinoma (PDAC).

In certain embodiments, systematic measurements of density of pancreatic tissues at each of the timed phases of contrast-enhanced pancreatic protocol CT scans were used in conjunction with the mathematical model to derive a mass transport parameter that quantified the area under the enhancement curve (AUC) for patients with PDAC. In certain embodiments, a “normalized AUC ratio” is defined as the post-therapy AUC (4-8 weeks after treatment) divided by the pre-therapy AUC. This parameter was correlated with local tumor control in patients treated on two previously published prospective trials. The first trial originally included 48 patients with locally advanced PDAC treated with radiation to 50.4 Gy with concurrent bevacizumab and capecitabine. The second trial originally included 69 patients with locally advanced PDAC treated with induction cetuximab, gemcitabine, and oxaliplatin, followed by radiation to 50.4 Gy with concurrent cetuximab and capecitabine. A total of 84 patients (36 from trial 1 and 48 from trial 2) had both post- and pre-therapy pancreatic protocol CT scans to analyze. The others did not have pancreatic protocol CT scans for one or both of the tests.

There were 30 patients with clinical and radiographic evidence of local progression. The 2 year local control rate was 50% for all patients. The normalized AUC ratio was discovered to significantly correlate with local progression-free survival (HR 1.81, 95% CI 1.01 to 3.03, p=0.048). Furthermore, patients with a measureable decrease in tumor mass transport (normalized AUC ratio<1) after chemoradiation had significantly better local control (86% with tumor control at 2 years) compared to those without a decrease in tumor mass transport (normalized AUC ratio≧1, 34% with tumor control at 2 years, p=0.002). As a continuous or discrete variable (with a cutoff of 1), the normalized AUC ratio correlated with local progression-free survival, independent of therapy regimen, change in tumor size after therapy, and receipt of curative-intent surgery.

Thus, after cytotoxic therapies, decreased enhancement in human PDAC tumors correlated with improved local tumor control. This phenomenon can be quantified using the systematic methodology and mathematical model according to certain embodiments of the invention. This method may serve as an early readout of response to new therapies, which may accelerate evaluation of promising therapies and enable rational management decisions.

In certain embodiments CT measurements may be used for esophageal cancer. In a dataset of 173 patients with distal adenocarcinoma of the esophagus treated with preoperative chemoradiation, measurements of enhancement of the esophageal tumor and normal esophagus were performed. Using the same principles as developed in human pancreatic cancer, a transport parameter normalized AUC was developed. This parameter is correlated with pathological response and overall survival.

In certain embodiments, this measurement of normalized AUC can be used as an input to other mathematical models. For example, a diffusion-based model of cell kill (Pascal et al. 2013 PNAS 110:14266-14271) from chemotherapy was applied to the measurements of CT enhancement in esophageal cancer. This diffusion-based model is designed to use measurements of mass transport to predict the cancer cell kill. Using normalized AUC and pathological response as inputs to the model, least-squares regression was used to derive estimates of the blood vessel radius (rb) and characteristic length between blood vessels (L) for the entire group. These parameters were then used to calculate a prediction of Fₖᵢₗₗ based on normalized AUC for each individual patient.

The results show that the model transforms the CT-based enhancement values non-linearly, providing an estimate of Fₖᵢₗₗ from the cytotoxic therapy. The model estimates of Fₖᵢₗₗ correlated with the pathological response measurements of the pathologist with relatively low error (linear regression p<0.0001 with root mean square error 0.05; Spearman rank-order correlation 0.28, 95% CI 0.14 to 0.42, p=0.0001), and these estimates of Fₖᵢₗₗ also correlated with overall survival on univariate and multivariate analyses.

The methods of the invention have been applied to pancreatic cancer and to esophageal cancer, were found to predict patient outcomes. As one of ordinary skill in the art will appreciate, this method can be applied in other solid tumors, including, for example, colorectal liver metastases and primary liver tumors like hepatocellular carcinoma.

### EXAMPLES

Methods

Development and Validation of a Novel Mass Transport Model for Human Pancreatic Protocol CT Scans.

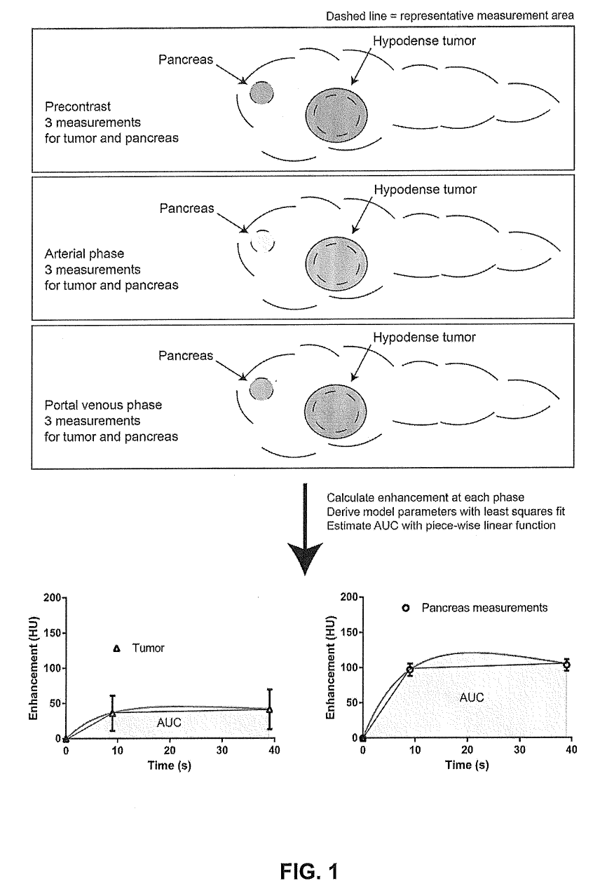

Mathematical modeling of the changes in enhancement of the tissues (Hounsefield Units [HU]) at sequential time points during the pancreatic protocol (pre-contrast, arterial, and portal-venous phases) could quantify the mass transport properties of individual tumors, including PDAC tumors (FIG. 1).

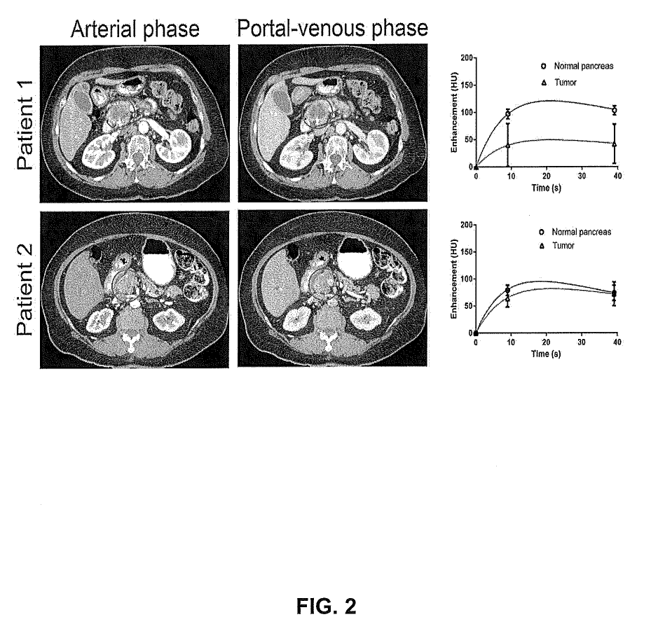

Multiple, systematic measurements obtained during the pancreatic protocol CT were used in a novel mathematical model to yield phenomenological parameters of mass transport that describe qualities of the pancreatic tissue (normal and tumor) and its surrounding vasculature (FIG. 2).

The transport model includes an ordinary differential equation describing the variable density Y(t) (HU) in the tissue as a function of time t resulting from transfer of contrast agent molecules through the vessel walls at a rate R (s⁻¹) and clearance rate from the vasculature R_{c} (s⁻¹):

dY/dt=R*(Y^{V}ₘₐₓ*exp(−R_{c}*t)−Y) (1)

where Y^{V}ₘₐₓ, represents the (imposed) level of density within the microvasculature. Eq. (1) is solved for initial condition Y(0)=0, giving the solution for the variable density:

Y(t)=Y^{V}ₘₐₓ*R*[exp(−R_{c}*t)−exp(−R*t)]/[R−R_{c}] (2)

Two other model parameters can be derived from the intrinsic variables (R, R_{c}, and Y^{V}ₘₐₓ) of the model, including a maximum enhancement of the tissue (Y^{T}ₘₐₓ) and an initial influx rate of contrast (R₀). The model function can also be integrated over time to give an area under the curve (AUC).

Model Assumptions

For the model, the microvasculature enhancement (Y^{V}(t)) is described by a first order decaying exponential at a rate R_{c}:

Y^{V}(t)=Y^{V}ₘₐₓ*exp(−R_{c}*t) (3)

Time zero for the model is defined as the time when Y^{V}(t) is maximum (i.e., Y^{V}(0)=Y^{V}ₘₐₓ). At M. D. Anderson, bolus tracking of the aorta has been used since 2006, whereby a value of 100 HU in the aorta triggers the countdown to start the arterial phase scan (16 s later for a 16 detector scanner and 20 s later for a 64 detector scanner). This bolus tracking method improves the chances of observing differences in contrast uptake in pancreatic tissues by reducing differences in cardiac output for patients. It was assumed that the model t=0 s occurred halfway between the bolus trigger of 100 HU and the beginning of the arterial phase, which would allow the contrast bolus to go through the cardiac circulation and reach the supplying vasculature of the pancreatic tissues. For patients who had pancreatic protocol CTs prior to 2006, the timing in relation to the start of contrast infusion was the same as those who received scans after 2006. Sensitivity analyses were performed to determine the effect of slight variations in the timing of the tests on the observed correlations. The model also assumes that the contribution of recirculation of contrast to the enhancement of the tissues is negligible during the test, as the arterial and portal-venous phases are completed in just over one minute after the start of contrast infusion.

Derivation of Model Parameters

The model parameters were estimated for each patient by performing least-square fits of the solution of the model for the variable density Eq. (2) to the three time-point CT measurements (in HU) at t=0 s (Y(0)=0 for all patients because precontrast density was subtracted), t=tₐᵣₜₑᵣᵢₐₗ (tₐᵣₜₑᵣᵢₐₗ=8 s for 16 detector scanner and 10 s for 64 detector scanner using arterial phase density measurement minus precontrast phase density measurement in same tissue region of patient), t=tᵥₑₙₒᵤₛ (tᵥₑₙₒᵤₛ=38 s for 16 detector scanner and 40 s for 64 detector scanner using portal-venous phase measurement minus precontrast phase density measurement in same tissue region of patient). The model definitions for tₐᵣₜₑᵣᵢₐₗ and tᵥₑₙₒᵤₛ correspond to 40 s and 70 s after the start of contrast infusion, which are traditionally in the range of ideal times for acquisition (36).

Two important additional variables can be derived from the model. Note that neither of these can actually be measured. The first variable is the initial “slope” or initial time derivative, R₀, of the predicted tissue density profile:

At t=0s, dY/dt=R₀=Y^{V}ₘₐₓ*R (3)

The second variable is the predicted “maximum value” of density attained within tissue (Y^{T}ₘₐₓ), which may happen outside of the time-window of the CT measurements:

At t=log(R/R_{c})/(R−R_{c}), dY/dt=0, and Y^{T}ₘₐₓ=Y^{V}ₘₐₓ*(R/R_{c})^{(-Rc/[R-Rc])}. (4)

Note that, as expected, for all t, Y(t)≦Y^{T}ₘₐₓ<Y^{V}ₘₐₓ as the value of density in the tissue cannot exceed the initial amount delivered to the microvasculature from the aorta.

The parameter AUC represents the time integral of the model-predicted enhancement function (Eq. 2) from time t=0 s to t=tᵥₑₙₒᵤₛ. The appropriateness of the model was tested by approximating AUC using a simple linear piece-wise function. Essentially, AUC was estimated as the sum of the area of a triangle and a trapezoid. In addition to showing the appropriateness of the model, this estimate of AUC is important for the clinical translation of this CT analysis, as the calculation is straightforward and can be done at any institution without additional algorithms or software.

CT Measurements

All analyses were performed on patients who were part of MD Anderson institutional review board-approved protocols. Enhancement (defined as post-contrast density minus pre-contrast density) of pancreatic tumors shows considerable heterogeneity. Multiple systematic measurements of the visualized tumors were used. Three HU measurements from the hypodense region of the tumor were made. The same regions were sampled at each phase of the pancreatic protocol CT. Similarly, 3 measurements from the normal pancreas that was not obstructed by the tumor were made during each phase of the CT. The averages for the tumor and normal pancreas were used to derive mass transport properties using Eq. 2.

Clinical Trial of Intraoperative Gemcitabine Infusion

Patients

Twelve patients with PDAC were enrolled on an institutional review board-approved (IRB) protocol. Written informed consent was obtained from all patients. All patients had cytologic or histologic proof of adenocarcinoma of the pancreas prior to treatment. Patients were staged with a physical exam, chest radiography, and contrast-enhanced computed tomography and only potentially resectable patients were eligible as determined by the operative surgeon per previous criteria. There was no upper age restriction and patients with Karnofsky performance status greater than 70 were eligible. All patients required adequate renal, hepatic, and bone marrow function based on preoperative lab testing and could not have other significant comorbidities that precluded surgical intervention.

Intraoperative Procedure

Pancreatic resection was performed using standard techniques and major surgical complications were defined as previously described. Gemcitabine was administered intravenously at 500 mg/m² for the first 2 patients (per IRB safety recommendations) and then subsequently at 1000 mg/m² with an infusion pump at a fixed dose rate of 10 mg/m²/min (over 50 min and 100 min respectively) and was delivered at the start of operation and concluded prior to specimen removal. Serum samples were collected at regular intervals during the operation to determine gemcitabine pharmacokinetics.

Toxicity Monitoring

The MD Anderson Data and Safety Monitoring Board oversaw the study. Drug toxicities were evaluated according to Common Terminology Criteria for Adverse Events version 3.0. Daily postoperative labs including absolute neutrophil counts (ANC) and platelet counts were drawn. Patients received full supportive care and the use of growth factors was permitted for myelosuppression (ANC<0.5×10⁹). There were 3 patients with grade III neutropenia or leukopenia, and 3 patients with grade II neutropenia. All cytopenias were transient with nadir at approximately postoperative day 6. No significant thrombocytopenia occurred in any patients. There were 2 patients given GM-CSF empirically and there were no infectious complications in the whole cohort.

Pathological Analysis

Immediately after specimen collection, tumors were bivalved by a surgical pathologist and 4.0 mm punch biopsies were taken from the outer and inner portions of the tumors (median 4 samples per tumor) as well from as the normal pancreas. The punch biopsies were sent for quantitative analysis of the gemcitabine metabolite incorporation into the DNA of the cells within the pancreatic tumor (i.e., cancer and stromal cells) and normal pancreas (Advion BioServices, Ithaca, N.Y.). The tumor and normal pancreatic tissue surrounding each punch biopsy site were submitted for histologic confirmation of the tissue type (tumor vs. normal) and assessment of stromal score by hematoxylin and eosin as well as Masson's trichrome stains, hENT1 (percentage of cells with staining intensity relative to lymphocyte control of 0 [no staining], 1+[low staining], 2+[equivalent staining], or 3+[high staining], MBL International, Woburn, Mass., and Ki67 (percentage of cells with staining, MBL International, Woburn, Mass.).

Intraoperative Gemcitabine Infusion Clinical Trial

In this study, patient eligibility, intraoperative gemcitabine infusion procedure, sampling and measurement technique for gemcitabine incorporation, and histological analysis were performed as described above. Briefly, patients with previously untreated, resectable pancreatic cancer were eligible for the trial. Patients were taken to the operating room, and after being deemed resectable by the surgeon, gemcitabine was infused intravenously through a peripheral line at a rate of 10 mg/m²/min to a total dose of 1000 mg/m² for all patients, except the first two. These two initial patients received 500 mg/m², based on IRB recommendations to ensure safety of the protocol. The blood supply to the pancreas was maintained until the specimen was ready for complete removal and after all gemcitabine was infused. The specimen was immediately taken to an adjacent surgical pathology suite for sampling.

Tumor Sampling for Gemcitabine Incorporation

The pancreatectomy specimens were evaluated by a pathologist. Multiple biopsy samples from the tumor and adjacent normal pancreas were taken with a 4 mm punch biopsy. The locations of the tumor biopsy samples that were used to quantify gemcitabine incorporation were recorded as either inner or outer tumor. Generally, the outer and inner tumor portions were designated by visual inspection and palpation at the time of tumor sampling by the pathologist. Typically, the outer tumor was identified as the outer 3-5 mm of the tumor, and the inner tumor was anything inside this rim. These specimens were then processed as previously described for quantitative analysis of gemcitabine DNA incorporation (Advion Biosciences). Proper calibration of the gemcitabine assay was performed.

CT Analysis

The pancreatic protocol CT scan is a diagnostic test for patients with pancreatic cancer, where iodine-based contrast is injected intravenously at a fixed rate. The test usually consists of a pre-contrast, an arterial phase (35-40 seconds after starting contrast infusion) and a portal-venous phase (65-70 seconds after starting contrast infusion). The CT acquisition is usually at a high resolution (0.6 to 2.5 mm slice thickness), enabling visualization of most pancreatic tumors due to differential contrast enhancement relative to the surrounding normal pancreas tissue. The pre-operative CT images were imported into Pinnacle 9.6 (Varian Medical Systems, Palo Alto, Calif.) for image registration of the pre-contrast, arterial phase, and portal venous phase of the pancreatic protocol CT scan for each patient. After the registration, three independent medical doctors delineated the aorta at the level of the celiac artery, normal pancreas, and pancreatic tumor. The general guidelines for radiographic segmentation of the tumor and normal pancreas were to concentrate on the tissue component (i.e., hypodense solid tumor and parenchyma of the normal pancreas, respectively), while avoiding the pancreatic duct, the surrounding fat space of the pancreas, and artifact from metal. Additionally, the segmentation of the solid tumor avoided enhancing portions of the pancreas parenchyma by 2-4 mm.

The outer portion of the tumor was also segmented to evaluate intra-tumoral heterogeneity in the transport properties, compared to the inner portion of the tumor. The outer and inner radiographic segmentation of the tumors were performed in retrospective review of the patients' images. The definitions for inner and outer tumor from the CT scan followed the same methodology as the pathology sampling for inner and outer tumor: the outer tumor was identified as the outer 3-5 mm of the visualized tumor, and the inner tumor was anything inside this rim.

The segmented structures (aorta, pancreas, tumor) were analyzed for density, measured in HU, within the delineated volume of interest. The volumetric mean of the HU at each phase of the scan was used to calculate the mass transport properties of each structure, providing an area under the enhancement curve (volumetric AUC), as described in the above mathematical model of transport.

Statistical Analysis

JMP 10.0 (SAS Institute) was used to perform all statistical analyses. All data were tested for normal distribution using the D'Agostino and Pearson omnibus normality test, where a p value greater than 0.05 was considered to pass the normality test. The Mann Whitney (Wilcoxon) test was used to compare the distributions between groups, and the Spearman's rank-order test was used for non-parametric correlation analysis, as appropriate. Linear regression (analysis of variance [ANOVA]) was used for correlations as long as normal distribution assumptions were met. All linear regression analyses are shown with the linear curve fit and 95% confidence interval (CI) shaded in blue. Survival curves were constructed using the Kaplan-Meier method. Cox proportional hazards model was used for univariate and multivariate survival analyses. Variables were included in the multivariate Cox proportional hazards model if the P value was 0.15 or less, or if variables have previously been demonstrated to influence outcome. A P value less than 0.05 was considered significant for all analyses.

### Results

Pre-therapy pancreatic protocol CTs from 176 patients with localized primary PDAC were analyzed using the methods described above. Three patient cohorts were studied: 12 who received intravenous (IV) infusion of gemcitabine during tumor resection on clinical trial (11 of whom had evaluable CTs); 110 who received gemcitabine-based chemoradiotherapy for potentially resectable PDAC; and 55 who received upfront tumor resection. The group of 55 patients served as the learning dataset in model development, providing a range of constraints for the parameters R and R_{c} for analysis of the other patient sets.

Image analysis identified a broad range of enhancement patterns for the patients, indicating that the pancreatic protocol CT can be used not only for diagnostic purposes but also for derivation of the physical properties of each patient's unique tumor. For normal pancreas tissue, AUC significantly correlated with the arterial phase enhancement in the aorta at the level of the celiac artery for each patient. Thus, the measurements and modeling of the behavior of the normal pancreatic tissue accounted for any differences in contrast dose and patient physiology (e.g., cardiac output). Similar analysis of the tumor tissues showed greater variability than those found in normal pancreas. The transport properties of pancreatic tumors and normal pancreas were significantly different, showing that the tissues can be quantitatively differentiated.

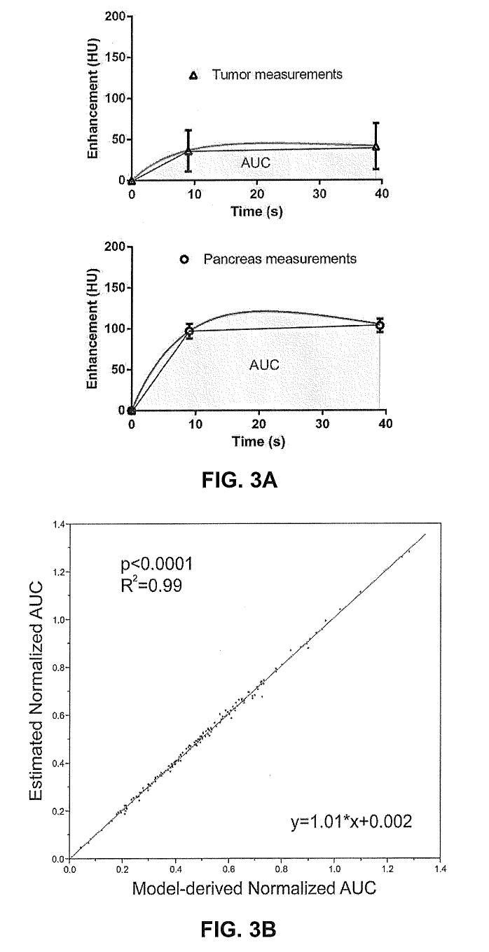

To assess the appropriateness of the developed model, the model-derived AUC were compared with an estimate of AUC using a simple piece-wise linear function. There was a 1:1 linear relationship between the estimated and the model-derived AUC parameter (FIG. 3). Moreover, the simple piece-wise linear approximation can be easily translated to clinical practice, as only a straightforward calculation is necessary.

Multi-Scale Transport Factors Influence Delivery of Gemcitabine to Human PDAC Cells.

A first-in-kind, prospective clinical trial (Koay et al., 2014 Journal of Clinical Investigations, volume 124, pages 1525-36) was conducted in which gemcitabine was intravenously infused during curative resection of 12 patients with localized primary PDAC. The objective was to determine whether transport-related factors influence gemcitabine incorporation into cellular DNA. The methods to quantitatively measure gemcitabine incorporation into DNA of cells in the tumor, blood samples, and other tissue were developed. Gemcitabine pharmacokinetics and hematological toxicity were similar for all patients. Despite similar intravascular pharmacokinetics and well-controlled infusion conditions, levels of gemcitabine incorporation into DNA of cells in the tumors and normal pancreas were highly variable among patients. Notably, gemcitabine incorporation in the tumors ranged from sub- to supra-normal pancreas levels.

The variability of gemcitabine incorporation in tumors among the patients could be explained by mass transport phenomena. In all correlations of transport phenomena, normalization of one variable in the correlation was important (e.g., normalized dependent variable vs. unnormalized independent variable, or vice versa). Using appropriate measurements in normal pancreas as the normalization factor reduced variability in the correlations. Variability may stem from biological differences among patients and minor differences in the acquisition timing of the CT scan.

Because extensive desmoplasia is a common feature of PDAC that reflects a putative stromal barrier to gemcitabine delivery and also impairs vasculature function, it was hypothesized that the stromal amount in the specimens would influence gemcitabine delivery. On initial evaluation, stromal score by itself did not correlate with tumor gemcitabine incorporation. Considering that expression of hENT1, a transporter of gemcitabine, correlates with outcome after adjuvant gemcitabine therapy in patients with PDAC and the initial multi-scale mass transport hypothesis, it was hypothesized that hENT1 scoring would improve correlations between stromal score and normalized gemcitabine incorporation. Patients were first ranked by hENT1 staining intensity and then assigned “high” and “low” designations down the ranked order. Subsequently, correlations between stromal score and normalized gemcitabine incorporation were assessed for these two hENT1 groups (high vs. low). Ultimately, five patients with high hENT1 staining and seven patients with low hENT1 staining were identified. Although the semi-quantitative nature of hENT1 intensity scoring limits this methodology, the final classification was similar to previous work. The hENT1 scores significantly correlated with normalized gemcitabine incorporation. Consistent with the multi-scale mass transport hypothesis, stromal score inversely correlated with normalized gemcitabine incorporation, after accounting for the hENT1 score.

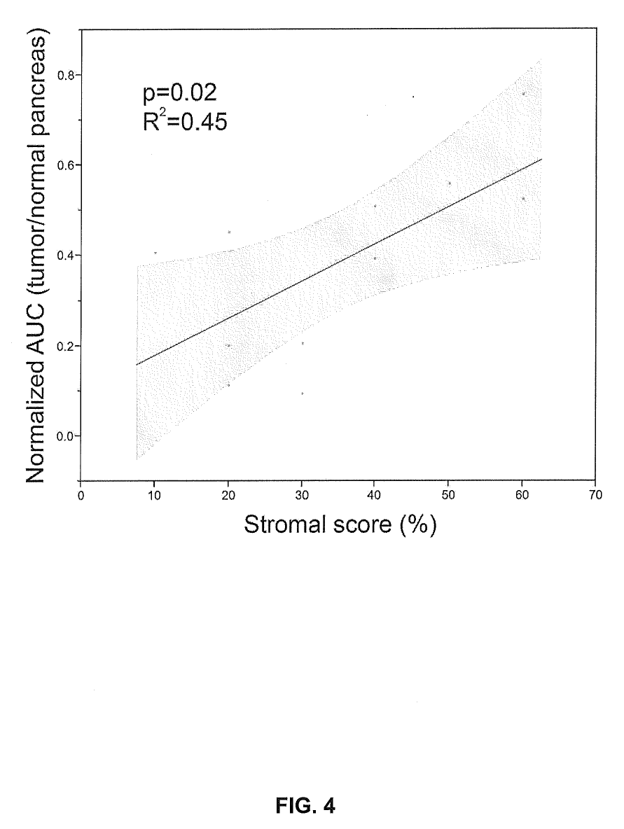

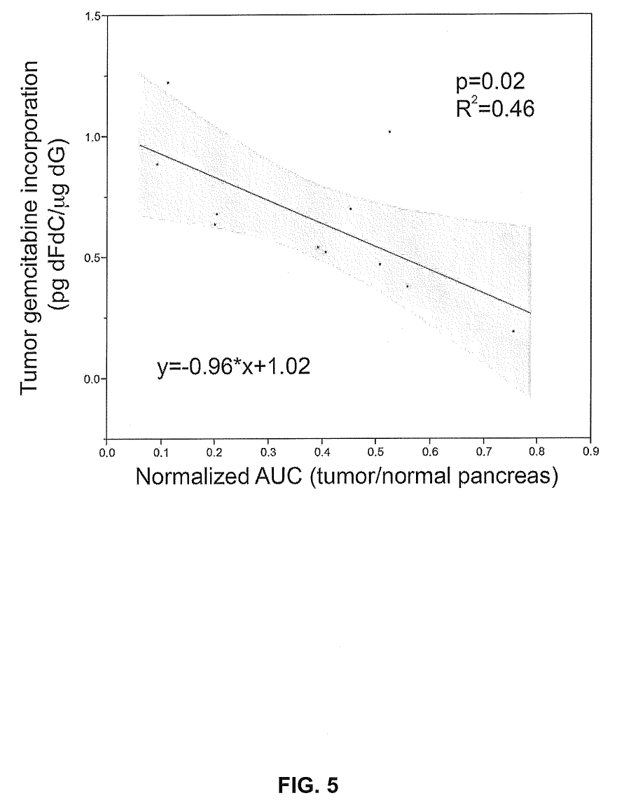

Furthermore, it was hypothesized that the CT-derived parameters would reflect tumor pathology and correlate with gemcitabine incorporation. Mass transport parameters were derived for 11 of 12 patients who had pre-therapy pancreatic protocol CTs, and significant correlations between the CT-derived parameters and tumor stromal score within the surgical specimen were noted (FIG. 4). Moreover, normalized CT-derived parameters also inversely correlated with tumor gemcitabine incorporation (FIG. 5). Cellular proliferation was considered another possible determinant of gemcitabine incorporation, but a significant correlation with Ki67 score was not found.

Inter-Patient and Intra-Tumoral Heterogeneity in Gemcitabine Delivery are Described by Transport Properties (Koay et al., 2014 Physical Biology, Volume 11, Page 065002).

The median number of tumor samples for the clinical trial was 4 per patient (range 2 to 6). The median number of samples from the outer portion of the tumor was 2 (range 1 to 3), and the median number of samples from the inner portion of the tumor was 2 (range 1 to 3).

As described in the previous section, there was significant inter-patient heterogeneity in gemcitabine DNA incorporation within the tumors. Here, we analyzed the samples according to the location of the sample to gauge intra-tumoral heterogeneity. A spectrum of intra-tumoral heterogeneity was observed in the 12 patients, with some patients having a lower proportional gemcitabine delivery to the inner portion of the tumor while others had proportionally higher delivery there, with the percentage of total gemcitabine DNA incorporation in the inner portion of tumors ranging from 38 to 74. Even within the designated inner and outer portions of each patient's tumor, variable patterns of gemcitabine DNA incorporation was observed, with standard deviations ranging from 6% to 87% of the mean. Similarly, there was a wide range of intra-tumoral heterogeneity in the transport properties. For example, some tumors showed negligible difference between the outer and inner portions of the tumor, while others showed about 200% difference in volumetric AUC between the outer and inner portions of the tumor.

Quantitative Segmentation can be Used to Measure Mass Transport Reproducibly.

Three different users independently segmented the aorta at the level of the celiac artery, normal pancreas, and pancreatic tumor. There was strong correlation between the volumetric AUC normalized by pancreas and volumetric AUC normalized by aorta (p<0.0001 for all observers). Additionally, there was high degree of agreement between the observers. For example, at the average value of volumetric AUC normalized by the pancreas for all patients, there was less than a 5% difference across the three observers in terms of the predicted value of volumetric AUC normalized by the aorta.

Mass Transport Properties Account for Heterogeneity in Gemcitabine Delivery.

Mass transport properties of the tumors were correlated with gemcitabine DNA incorporation in the inner and outer portions of the tumor, as well as an average of all of the measurements from the tumor. As discussed above, the amount of stroma and the expression of hENT1 influenced gemcitabine incorporation, supporting the idea of multi-scale transport dysregulation in pancreatic cancer. In this study, it was discovered that the CT-derived transport properties significantly correlated with the gemcitabine incorporation with or without accounting for hENT1 status. There was high inter-observer agreement in the correlations between transport properties and gemcitabine DNA incorporation (less than 5% differences in the predicted gemcitabine DNA incorporation at the average value of volumetric AUC normalized by aorta). The transport properties were correlated with gemcitabine DNA incorporation for all patients, for patients with low hENT1 scores, and for patients with high hENT1 scores. As above, low and high hENT1 scores were assigned by ranking staining intensities of hENT1 using immunohistochemistry. The correlation between CT-derived mass transport and gemcitabine DNA incorporation was seen on linear regression analysis as well as Spearman rank order correlation. Significant correlations were also seen after accounting for hENT1 expression. Indeed, the highest degree of correlation was seen for the outer portion of the tumor in patients with low hENT1 expression.

CT-Derived Transport Parameters Correlate with Response to and Survival after Gemcitabine-Based Therapy.

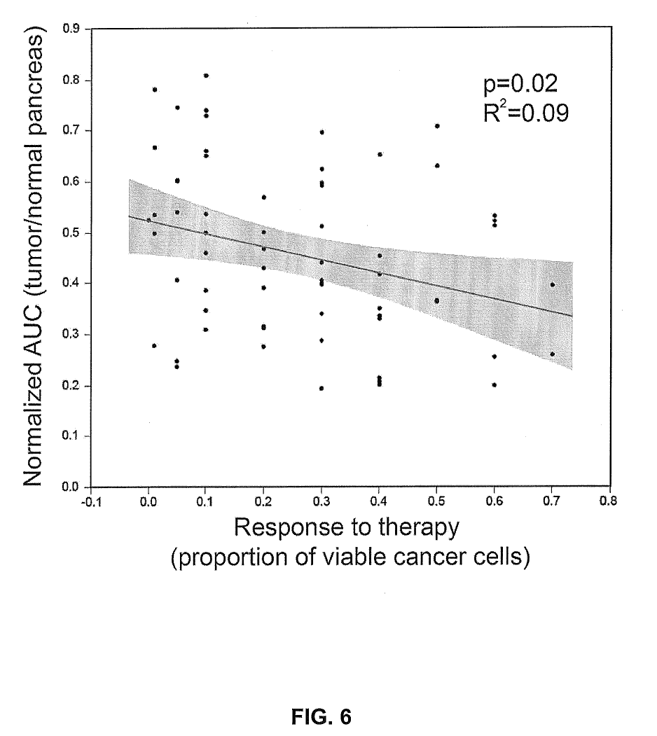

Because the clinical trial suggested physical mass transport properties could describe the variability in delivery of gemcitabine to tumor cell DNA, it was hypothesized that the transport properties could also describe the variable response to and outcome after gemcitabine-based therapies. To test this idea, CT parameters were correlated with pathological response and survival of patients with PDAC after preoperative chemoradiation (Evans et al. 2008 Journal of Clinical Oncology volume 26 pages 3496-502; Varadhachary et al. 2008 Journal of Clinical Oncology volume 26 pages 3487-95). Patients (110) who received gemcitabine-based chemoradiation and had evaluable pre-therapy CT scans from two prospective clinical trials were identified for potentially resectable PDAC. There were 66 patients from these two trials who did not have evaluable CT scans because the initial scan was either from another institution or not a pancreatic protocol CT. Of the 110 patients evaluated, 80 underwent a curative resection. The other 30 were unresectable after chemoradiation. Surgical resection and the CT-derived parameter normalized AUC were the only variables that significantly correlated with overall survival in the entire cohort of 110 patients on both univariate and multivariate analyses. A subgroup analysis of the 80 patients who underwent resection revealed that patients with pathologically involved nodes after chemoradiation had poorer grades of response (proportion of viable cells, 0.31±0.20) compared to patients with no pathologically involved nodes after neoadjuvant therapy (0.20±0.16, p=0.02); no other clinical or treatment-related factors correlated with pathological response or survival, except Normalized AUC. Distinct CT signatures in patients who had complete pathological responses were observed compared with those with poor responses to therapy, and the normalized CT-derived parameter AUC directly correlated with pathological response (FIG. 6). As previously observed (Chatterjee et al. 2012 Cancer volume 118 pages 182-90), patierits with better grades of pathological responses (i.e., fewer viable cells after therapy) had improved prognosis, and as response correlated directly with normalized AUC, patients with higher values of normalized AUC also had improved prognosis. Multivariate analysis of the 80 patients who underwent resection confirmed that normalized AUC was an independent predictor of overall survival.

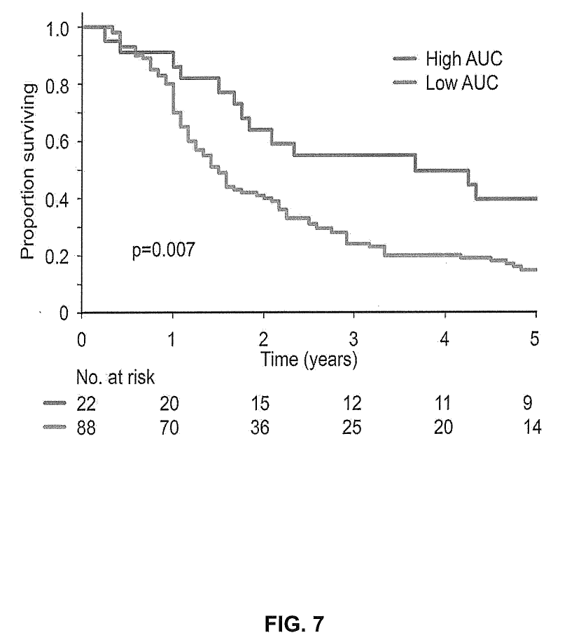

An exploratory partitioning analysis identified a cut-off for normalized AUC of 0.6. Applying this cut-off to the entire cohort of 110 patients showed that patients with “high” normalized AUC had significantly better outcome (40% survival rate at 5 years) compared to patients with “low” normalized AUC (15% survival rate at 5 years), independent of whether the patients had curative-intent surgery (FIG. 7). Additionally, the same cut-off for normalized AUC remained a significant predictor of survival in the 80 patients who underwent surgery, independent of margin status and lymph node involvement.

CT-Based Transport Parameters Correlate with Stromal Content and Survival in Patients Who Undergo Upfront Surgery.

Patients (n=101) with early stage PDAC who underwent upfront surgery were analyzed using the methods of the invention. The median age was 64 years (25-84). 81% of the patients had N1 disease (positive lymph nodes). 84% of patients received adjuvant chemotherapy. 28% received adjuvant chemoradiation. The same measurement of normalized AUC was applied to the pre-surgical scans of these patients. The amount of stroma on the surgical specimens, as above. It was discovered that there was significant association between normalized AUC and the amount of stroma, consistent the finding that higher normalized AUC correlated with greater amounts of stroma. Also consistent with the patients who received neoadjuvant therapy, it was discovered that patients who underwent upfront surgery for localized PDAC and who had higher values of normalized AUC had improved prognosis, as normalized AUC was associated with overall survival (p=0.03).

Changes in Mass Transport and Correlation with Local Control in Pancreatic Cancer.

Transport properties were derived, using methods described above, from the pre-therapy CT scans for 101 patients who participated in two prospective clinical trials of locally advanced, unresectable PDAC (48 of 48 patients from one study [Crane et al. 2006 Journal of Clinical Oncology volume 24 pages 1145-51], and 53 of 69 from the other [Crane et al. 2011 Journal of Clinical Oncology volume 29 pages 3037-3043]). Patient age, therapeutic regimen, CA19-9 level, and tumor size had no correlation with overall survival. There was a significant correlation between overall survival and the pre-therapy transport properties (continuous variable HR for death 0.49, 95% CI 0.23 to 0.97, p=0.04). This finding was also significant on multivariate analysis, accounting for 9 patients who underwent curative-intent surgery. This demonstrates the clinical significance of the mass transport properties in another large dataset of patients, this time with locally advanced PDAC treated with chemoradiation.

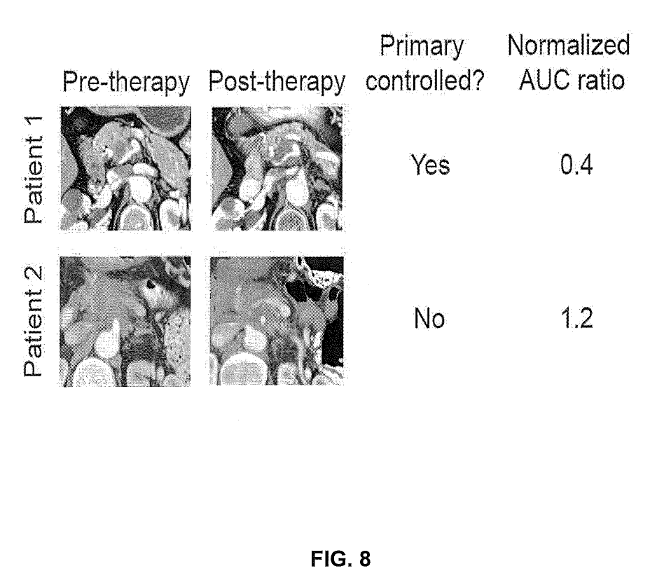

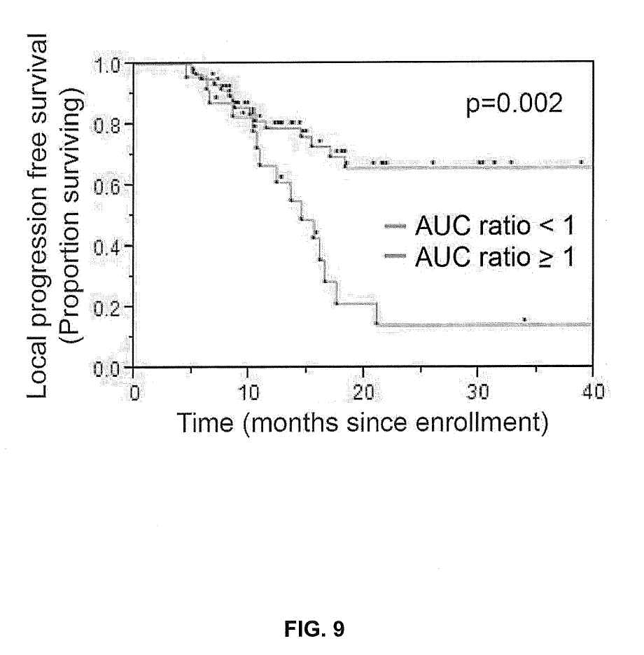

In these two trials of locally advanced PDAC, the CT-derived normalized AUC was quantified in the pretherapy and posttherapy scans (6 to 8 weeks after completion of chemoradiation) in 84 patients for whom both pre- and posttherapy scans were available. A parameter termed “normalized AUC ratio,” which represents the normalized AUC measured from the post-therapy scan divided by the normalized AUC measured from the pre-therapy scan, was defined (FIG. 8). It was discovered that normalized AUC ratio correlates with local progression free survival (LPFS) as a continuous variable (HR 1.81, 95% CI 1.01-3.03) or as a discrete variable (cutoff of 1, HR 3.01, 95% CI 1.45-6.25) on univariate and multivariate analyses, accounting for the patients who underwent surgery. Moreover, in 37 patients (19 had local progression) with post-induction chemotherapy scans, normalized AUC ratio (as a continuous variable) after induction chemotherapy also correlated with LPFS (HR 2.34, 95% CI 1.10-4.65, FIG. 9). Thus, effectiveness of cytotoxic therapy could be assessed weeks after therapy using the normalized AUC ratio.

Extension of Method to Esophageal Cancer.

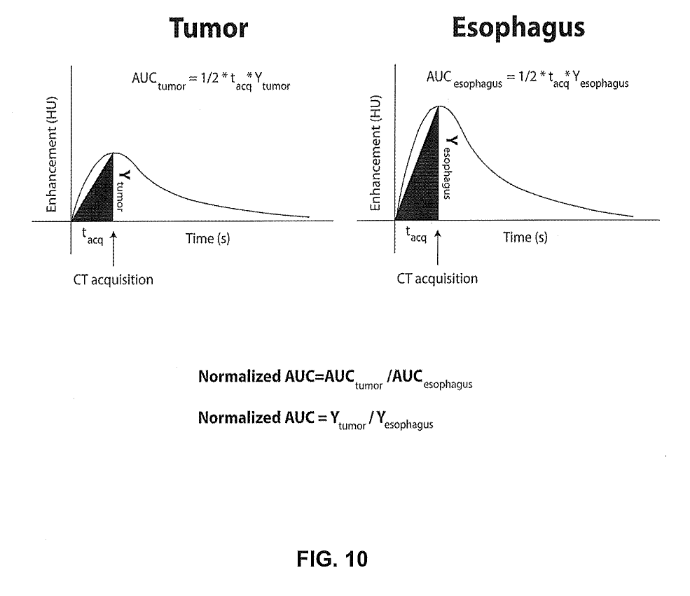

173 CT scans of patients who underwent neoadjuvant chemoradiation for distal esophageal cancer were analyzed. The described method of measuring AUC in the tumors by measuring the enhancement value in the tumor and the adjacent esophageal tissue was applied. Here, AUC is estimated by the equation for the area of a triangle (i.e., AUC=½ *t acq*Y tissue), where t acq is the time of acquisition of the scan and Y tissue is the enhancement value of the tissue (tumor or esophagus) measured in HU (FIG. 10). To calculate a normalized AUC for esophageal cancer, the AUC of the tumor is divided by the AUC of the esophagus. In this case, the equation simplifies to dividing the Y tumor by the Y esophagus. By doing this calculation, it was discovered that the normalized AUC for distal esophageal cancer also correlated with the pathological response to chemoradiation (p<0.01) and the overall survival of the patients (p=0.02).

While the disclosure is susceptible to various modifications and alternative forms, specific exemplary embodiments of the present invention have been shown by way of example in the drawings and have been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

1. A method for quantitatively describing a physical property of a tissue in a subject, comprising the steps of:
administering a contrast material to the subject;
obtaining radiology scans of the tissue at various time points including scans obtained prior to and after administering the contrast material;
using the radiology scans to obtain a plurality of density measurements;
detecting a change in the density measurements over time by applying a mathematical model of mass transport to describe the density measurement change, wherein the mathematical model comprises:
$$\frac{dy}{dt} = r \cdot ( {Y}_{max} \cdot {e}^{- {r}_{c} t} - Y ) ,$$
with dy/dt representing a variable density in the tissue as a function of time t, r representing a rate of transfer of the contrast material from micro-vasculature to the tissue, Y representing a density within the micro-vasculature, Yₘₐₓ representing a maximum density within the micro-vasculature, r_{c} representing a rate of clearance of the contrast material from the micro-vasculature.

2. The method of claim 1, wherein the radiology scans are computed tomography (CT) scans or magnetic resonance imaging (MRI) scans.

3. The method claim 1, wherein the radiology scans include precontrast, arterial, portal-venous, and delay.

4. The method of a claim 1, wherein the tissue comprises normal tissue and cancerous tissue.

5. The method of a claim 1 further comprising the step of measuring a chemical property of cells of the tissue.

6. The method of claim 5, wherein the chemical property comprises an expression level of a transporter for an agent of interest.

7. The method of a claim 1 further comprising the step of measuring uptake of a therapeutic agent by a cell of the tissue.

8. The method of a claim 1, wherein the tissue is pancreatic tissue.

9. The method of claim 1, wherein the tissue is esophageal tissue.

10. The method of a claim 1, wherein the tissue comprises normal tissue and a tumor.

11. The method of claim 8, wherein the pancreatic tissue comprises a pancreatic ductal adenocarcinoma and stromal content of the pancreatic ductal adenocarcinoma.

12.-13. (canceled)

14. The method of claim 1, wherein the mathematical model is solved for intimal condition y(0)=0 to arrive at a solution for the variable density.

15. The method of claim 1, wherein the solution for the variable density is:
$$\frac{dy}{dt} = {Y}_{max} \cdot r \cdot \frac{{e}^{- {r}_{c} t} - {e}^{- rt}}{r - {r}_{c}} .$$

16. The method of claim 1, wherein the initial time derivative is:
at t=0,Y(t)=Yₘₐₓ·r.

17. The method of claim 1, wherein a predicted maximum value of the density attained within the tissue (Y^{T}ₘₐₓ) is:
$$at t = \frac{log ( \frac{r}{{r}_{c}} )}{r - {r}_{c}} , \\ \frac{dy}{dt} = 0 and {Y}_{max}^{T} = {Y}_{max} \cdot {( \frac{r}{{r}_{c}} )}^{- \frac{{r}_{c}}{r - {r}_{c}}} .$$

18. A method to identify differences between normal tissue and abnormal tissue of one or more diagnostic scans, comprising:
describing a variable density
$$( \frac{dy}{dt} )$$
in a tissue as a function of time t resulting from transfer of contrast agent molecules through vessel walls, wherein the variable density
$$( \frac{dy}{dt} )$$
is:
$$\frac{dy}{dt} = {Y}_{max} \cdot r \cdot \frac{{e}^{- {r}_{c} t} - {e}^{- rt}}{r - {r}_{c}}$$
Yₘₐₓ represents a maximum density within micro-vasculature of the tissue, r represents a rate of transfer of the contrast agent molecules from micro-vasculature to the tissue, and r_{c} represents a rate of clearance of the contrast agent molecules from the micro-vasculature.

19. The method of claim 18, wherein the tissue comprises pancreatic ductal adenocarcinoma (PDAC) tissue.

20. The method of claim 18, wherein the diagnostic scans are computed tomography (CT) scans.

21. The method of claim 18, wherein the diagnostic scans are magnetic resonance imaging (MRI) scans.
